(19) United States
(12) Patent Application Publication
(10) Pub. No.: US 2018/0130557 A1
(43) Pub. Date: May 10, 2018

(54) ELECTRONIC DEVICE AND METHOD FOR RECORDING HEALTH

(71) Applicant: Hangzhou Mega Inno of Health Technology Co. Ltd, Hangzhou (CN)

(72) Inventors: CHIH-SIUNG CHIANG, New Taipei (TW); ZHAOHUI ZHOU, Santa Clara, CA (US); NENG-DE XIANG, Shenzhen (CN); PING-HAO LIU, New Taipei (TW); XUE-QIN ZHANG, Shenzhen (CN)

(21) Appl. No.: 15/721,645

(22) Filed: Sep. 29, 2017

(30) Foreign Application Priority Data
Oct. 12, 2016 (CN) 201610891422.1

(51) Int. Cl. G16H 40/67; A61B 5/0205; A61B 5/00; G16H 50/30; G16H 15/00

(52) U.S. Cl. CPC G16H 40/67; A61B 5/02055; A61B 5/6898; A61B 5/021; G16H 15/00; A61B 2560/0242; G16H 50/30

(57) ABSTRACT

A method for reading and recording physical condition of a user using an electronic device includes reading and recording physiological status of user when at least one collecting device of the electronic device is collecting physiological readings of a user. The readings are received by the electronic device, and a geographic location of the electronic device and environmental information of the geographic location are also acquired. A report and warning if necessary about physical condition of the user is generated by analyzing the status recorded and the environment information.

### CROSS-REFERENCE TO RELATED APPLICATIONS

This application claims priority to Chinese Patent Application No. 201610891422.1 filed on Oct. 12, 2016, the contents of which are incorporated by reference herein.

### FIELD

The subject matter herein generally relates to health management, and particularly to an electronic device and a method for recording health of a user using the electronic device.

### BACKGROUND

An electronic device can take measurements when a user does some activity. For example, when the user does a blood pressure test, the electronic device can read and record the blood pressure (e.g., 120/80) and the time (14:00, May 14, 2017). However, such measurements are too few for a deep analysis of health of the user.

### BRIEF DESCRIPTION OF THE DRAWINGS

Many aspects of the disclosure can be better understood with reference to the following drawings. The components in the drawings are not necessarily drawn to scale, the emphasis instead being placed upon clearly illustrating the principles of the disclosure. Moreover, in the drawings, like reference numerals designate corresponding parts throughout the several views.

FIG. 1 is a schematic diagram of one exemplary embodiment of electronic device.

FIG. 2 is a block diagram of one exemplary embodiment of the electronic device including a recording system.

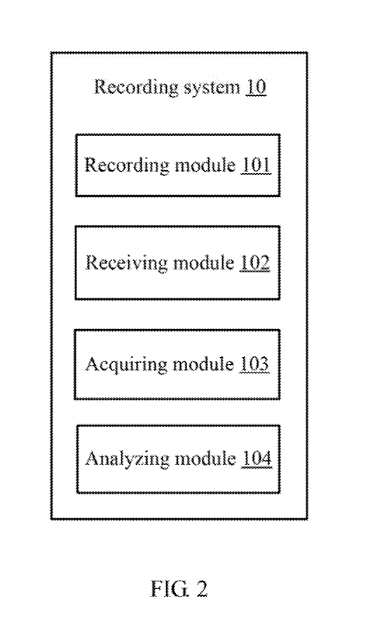

FIG. 3 illustrates a flow chart of an exemplary embodiment of a method for recording health of a user using the electronic device of FIG. 1.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The present disclosure, including the accompanying drawings, is illustrated by way of examples and not by way of limitation. It should be noted that references to “an” or “one” embodiment in this disclosure are not necessarily to the same embodiment, and such references mean “at least one.”

The term “module”, as used herein, refers to logic embodied in hardware or firmware, or to a collection of software instructions, written in a programming language, such as, Java, C, or assembly. One or more software instructions in the modules can be embedded in firmware, such as in an EPROM. The modules described herein can be implemented as either software and/or hardware modules and can be stored in any type of non-transitory computer-readable medium or other storage device. Some non-limiting examples of non-transitory computer-readable media include CDs, DVDs, BLU-RAY™, flash memory, and hard disk drives. The term “comprising” means “including, but not necessarily limited to”; it specifically indicates open-ended inclusion or membership in a so-described combination, group, series and the like.

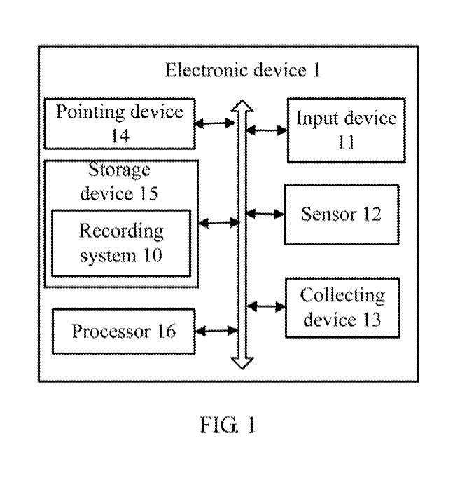

FIG. 1 is a block diagram of one embodiment of an electronic device 1. Depending on the embodiment, the electronic device 1 can include, but is not limited to, an input device 11, at least one sensor 12, at least one collecting device 13, a pointing device 14, a storage device 15, and at least one processor 16. The above components communicate with each other through a system bus. In at least one embodiment, the electronic device 1 can be a multi-parameter supervisor, a mobile phone, a personal computer, a tablet computer, a personal digital assistant, or any other suitable device. FIG. 1 illustrates only one example of the electronic device 1 that can include more or fewer components than illustrated, or have a different configuration of the various components in other embodiments. For example, the electronic device 1 further can include an electrical system, a sound system, an input/output interface, a battery, and an operating system.

In at least one exemplary embodiment, the input device 11 can be a display screen, an audio input device, a keyboard, and a mouse. The display screen is a multi-touch panel that can respond to touches. For example, the display screen can be a resistive touch screen, or a capacitive touch screen. The audio input device can be a microphone. In other exemplary embodiments, the input device can be a headset of the electronic device 1.

In at least one exemplary embodiment, the at least one sensor 12 can detect nature of environment around the electronic device 1. The at least one sensor 12 can include, but is not limited to, a temperature sensor and a humidity sensor. The temperature sensor can detect temperature of current environment, and the humidity sensor can detect humidity of the current environment.

In at least one exemplary embodiment, the at least one collecting device 13 can collect physiological readings of a user. The physiological readings can include, but are not limited to, blood pressure, electrocardiogram, blood glucose, body fat, body temperature, and blood oxygen level. The at least one collecting device 13 can be built into the electronic device 1 or can wired or wirelessly connect to the electronic device 1. The at least one collecting device 13 can include, but is not limited to, a blood pressure, an electrocardiogram equipment, a glucose meter, a body fat monitor, a thermometer, and an oximetry device. The blood pressure monitor can measure the blood pressure of the user. The electrocardiogram equipment can measure the electrocardiogram of the user. The glucose meter can measure the blood glucose of the user. The body fat monitor can measure the body fat of the user. The thermometer can measure the body temperature of the user. The oximetry device can measure the blood oxygen level of the user.

In at least one exemplary embodiment, the pointing device 14 can detect geographic location of the electronic device 1. The pointing device 14 can be a Global Positioning System (GPS), an Assisted Global Positioning System (AGPS), a BeiDou Navigation Satellite System (BDS), and a GLOBAL NAVIGATION SATELLITE SYSTEM (GLONASS). The geographic location can include longitude and latitude, and street address.

In at least one exemplary embodiment, the storage device 15 can be a memory device of the electronic device 1. In other exemplary embodiments, the storage device 15 can be a secure digital card, or other external storage device such as a smart media card. In at least one exemplary embodiment, the storage device 15 can store a recording system 10 of the electronic device 1.

The at least one processor 16 can be a central processing unit (CPU), a microprocessor, or other data processor chip that performs functions of the electronic device 1.

In at least one exemplary embodiment, the recording system 10 can include a recording module 101, a receiving module 102, an acquiring module 103, and an analyzing module 104. The modules 101-104 include computerized codes in the form of one or more programs that may be stored in the storage device 15. The computerized codes include instructions that can be executed by the at least one processor 16.

In at least one exemplary embodiment, the recording module 101 can record status when the at least one collecting device 13 is collecting the physiological readings of the user. The status show health of the user. The user may suffer from disorder or may be healthy. The status can acquire during daily life of the user. The electronic device 1 can generate health warning for a user by analyzing the status recorded. The status can include, but is not limited to, lifestyle information of the user, current environmental characteristics of user's life, current physical condition, and food the user eats.

In at least one exemplary embodiment, a display screen of the electronic device 1 can provide an interface for the user to input the status. For example, the user may input the lifestyle information as before dinner, at dinner, or after dinner, before sleep or after sleep, before exercise, doing exercise, or after exercise. The current environmental characteristics of life of the user can include some specialty environments. For example, when the user feels uncomfortable (e.g., through dizziness) in a room with a plant, the recording module 101 can record the plant as relevant to current environmental characteristics. The user may input the current physical condition of the user as feeling dizzy and tired. The user may input the food the user eats if the user feels uncomfortable after eating. For example, if the user feels uncomfortable after eating oatmeal, the recoding module 101 can record the oatmeal which was eaten. The user can measure the physiological readings using the electronic device 1 to analyze the effect of the oatmeal.

In at least one exemplary embodiment, the receiving module 102 can receive the physiological readings from the at least one collecting device 13.

In at least one exemplary embodiment, the acquiring module 103 can acquire the geographic location of the electronic device 1 from the pointing device 14 and the environment information of the geographic location. The environment information can include, but is not limited to, humidity, temperature, and weather (e.g., sunny). The acquiring module 103 can acquire the humidity from the humidity sensor, and acquire the temperature from the temperature sensor.

In at least one exemplary embodiment, the electronic device 1 further include a communication device (not shown). The electronic device 1 can connect to Internet through the communication device. The communication device can supply any conventional wired network, wireless network and the both. The wired network can be any category of conventional wired communications, for example, the Internet, local area network (LAN). The wireless network can be any category of conventional wireless communications, for example, radio, Wireless Fidelity (WiFi), cellular, satellite, and broadcasting. Exemplary suitable wireless communication technologies include, but are not limited to, Global System for Mobile Communications (GSM), General Packet Radio Service (GPRS), Code Division Multiple Access (CDMA), Wideband CDMA (W-CDMA), CDMA2000, IMT Single Carrier, Enhanced Data Rates for GSM Evolution (EDGE), Long-Term Evolution (LTE), LTE Advanced, Time-Division LTE (TD-LTE), High Performance Radio Local Area Network (HiperLAN), High Performance Radio Wide Area Network (HiperWAN), High Performance Radio Metropolitan Area Network (HiperMAN), Local Multipoint Distribution Service (LMDS), Worldwide Interoperability for Microwave Access (WiMAX), ZigBee, Bluetooth, Flash Orthogonal Frequency-Division Multiplexing (Flash-OFDM), High Capacity Spatial Division Multiple Access (HC-SDMA), iBurst, Universal Mobile Telecommunications System (UMTS), UMTS Time-Division Duplexing (UMTS-TDD), Evolved High Speed Packet Access (HSPA+), Time Division Synchronous Code Division Multiple Access (TD-SCDMA), Evolution-Data Optimized (EV-DO), Digital Enhanced Cordless Telecommunications (DECT) and others.

In at least one exemplary embodiment, the acquiring module 103 can acquire the weather from the Internet. The weather can include, but is not limited to, chance of rain, wind speed, apparent temperature, precipitation, air pressure, visibility, UV index, and air quality index.

In at least one exemplary embodiment, the analyzing module 104 can generate a report about physical condition of the user by analyzing the status recorded, the physiological readings, and the environment information.

In at least one exemplary embodiment, the analyzing module 104 can compare the physiological readings with parameter reference ranges and generate a result. The analyzing module 104 further can generate the report about the physical condition of the user by combining the generated result with the status recorded and the environment information. For example, the analyzing module 104 can sort and classify the status recorded to find out that an irregular mild headache of the user could be related or is related to a certain temperature and certain environmental characteristics. Then, the analyzing module 104 can prompt the user to avoid staying at a place for a long time with such temperature and such environmental characteristics.

In at least one exemplary embodiment, the analyzing module 104 further can analyze how the weather, the humidity, and the temperature affect the physiology of the user. For example, when the user stays at a place with low temperature and high humidity, the analyzing module 104 can generate report that the blood pressure of the user is low and the user will suffer lumbar and leg pain. Then, the electronic device 1 can prompt the user to take medication to increase the blood pressure or warn the user not stay at the place with low temperature and high humidity.

In at least one exemplary embodiment, the analyzing module 104 further can analyze whether a critical temperature and a critical humidity of a place can cause the user suffer a drop in good health. When the critical temperature and the critical humidity can cause such drop, the analyzing module 104 can warn the user not stay at the place with the critical temperature and the critical humidity.

In at least one exemplary embodiment, the analyzing module 104 further can compare the generated report with an old report to analyze changes of the physical condition of the user. For example, when the body health of the user becomes poor, the analyzing module 104 can prompt the user to change the lifestyle or environment according to the status recorded or the environment information.

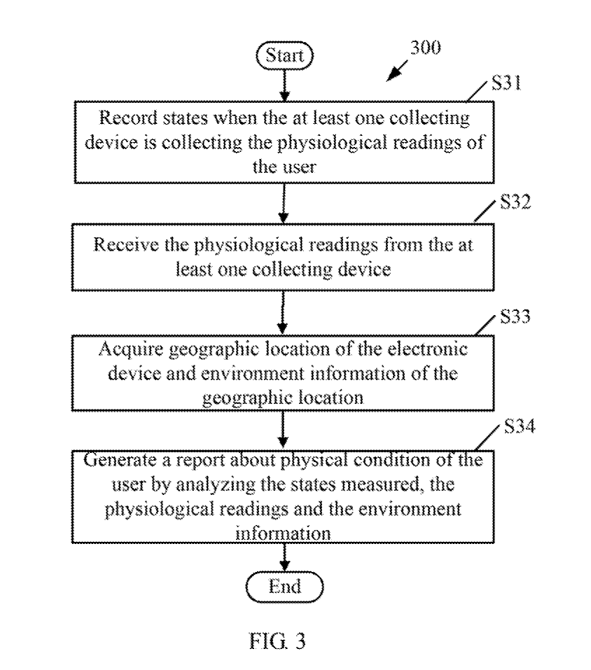

FIG. 3 illustrates a flowchart which is presented in accordance with an example embodiment. The exemplary method 300 is provided by way of example, as there are a variety of ways to carry out the method. The method 300 described below can be carried out using the configurations illustrated in FIG. 1, for example, and various elements of these figures are referenced in explaining exemplary method 300. Each block shown in FIG. 3 represents one or more processes, methods, or subroutines, carried out in the exemplary method 300. Additionally, the illustrated order of blocks is by example only and the order of the blocks can be changed according to the present disclosure. The exemplary method 300 can begin at block S31. Depending on the embodiment, additional steps can be added, others removed, and the ordering of the steps can be changed.

At block S31, the recording module 101 can record status when the at least one collecting device 13 is collecting the physiological readings of the user. The status can include, but is not limited to, lifestyle information of the user, current environmental characteristics of user's life, current physical condition, and food the user eats.

At block S32, the receiving module 102 can receive the physiological readings from the at least one collecting device 13. The physiological readings can include, but are not limited to, blood pressure, electrocardiogram, blood glucose, body fat, body temperature, and blood oxygen level.

At block S33, the acquiring module 103 can acquire the geographic location of the electronic device 1 and the environment information of the geographic location. The acquiring module 103 can acquire the geographic location from the pointing device 14. The environment information can include, but is not limited to, humidity, temperature and weather.

At block S34, the analyzing module 104 can generate a report about physical condition of the user by analyzing the status recorded, the physiological readings, and the environment information.

It should be emphasized that the above-described embodiments of the present disclosure, including any particular embodiments, are merely possible examples of implementations, set forth for a clear understanding of the principles of the disclosure. Many variations and modifications can be made to the above-described embodiment(s) of the disclosure without departing substantially from the spirit and principles of the disclosure. All such modifications and variations are intended to be included herein within the scope of this disclosure and protected by the following claims.

What is claimed is:

1. An electronic device comprising:
at least one collecting device;
a storage device;
at least one processor; and
the storage device further storing one or more programs that, when executed by the at least one processor, cause the at least one processor to:
record status when the at least one collecting device is collecting physiological readings of a user;
receive the physiological readings from the at least one collecting device;
acquire a geographic location of the electronic device and environment information of the geographic location; and
generate a report about physical condition of the user by analyzing the status recorded, the physiological readings, and the environment information.

2. The electronic device according to claim 1, wherein the status comprise lifestyle information of the user, current environmental characteristics of user's life, current physical condition, and food the user eats.

3. The electronic device according to claim 1, wherein the physiological readings comprise blood pressure, electrocardiogram, blood glucose, body fat, body temperature, and blood oxygen level.

4. The electronic device according to claim 3, wherein the report about the physical condition of the user is generated by:
generating a result by comparing the physiological readings with parameter reference ranges; and
combining the result with the status recorded and the environment information.

5. The electronic device according to claim 1, wherein the environment information comprises humidity, temperature, and weather.

6. The electronic device according to claim 5, wherein the humidity is acquired from a humidity sensor of the electronic device;
the temperature is acquired from a temperature sensor of the electronic device; and
the weather is acquired from internet through a communication device of the electronic device.

7. A recording method applied in an electronic device, the electronic device comprising at least one collecting device, the method comprising:
recording status when the at least one collecting device is collecting physiological readings of a user;
receiving the physiological readings from the at least one collecting device;
acquiring a geographic location of the electronic device and environment information of the geographic location; and
generating a report about physical condition of the user by analyzing the status recorded, the physiological readings, and the environment information.

8. The method according to claim 7, wherein the status comprise lifestyle information of the user, current environmental characteristics of user's life, current physical condition, and food the user eats.

9. The method according to claim 7, wherein the physiological readings comprise blood pressure, electrocardiogram, blood glucose, body fat, body temperature, and blood oxygen level.

10. The method according to claim 9, wherein the report about the physical condition of the user is generated by:
generating a result by comparing the physiological readings with parameter reference ranges; and
combining the result with the status recorded and the environment information.

11. The method according to claim 7, wherein the environment information comprises humidity, temperature, and weather.

12. The method according to claim 11, wherein the humidity is acquired from a humidity sensor of the electronic device;
the temperature is acquired from a temperature sensor of the electronic device; and
the weather is acquired from internet through a communication device of the electronic device.

13. A non-transitory storage medium having stored thereon instructions that, when executed by a processor of an electronic device, causes the processor to perform a recording method, the electronic device comprising at least one collecting device, the method comprising:
recording status when the at least one collecting device is collecting physiological readings of a user;
receiving the physiological readings from the at least one collecting device;
acquiring a geographic location of the electronic device and environment information of the geographic location; and
generating a report about physical condition of the user by analyzing the status recorded, the physiological readings, and the environment information.

14. The non-transitory storage medium according to claim 13, wherein the status comprise lifestyle information of the user, current environmental characteristics of user's life, current physical condition, and food the user eats.

15. The non-transitory storage medium according to claim 13, wherein the physiological readings comprise blood pressure, electrocardiogram, blood glucose, body fat, body temperature, and blood oxygen level.

16. The non-transitory storage medium according to claim 15, wherein the report about the physical condition of the user is generated by:
generating a result by comparing the physiological readings with parameter reference ranges; and
combining the result with the status recorded and the environment information.

17. The non-transitory storage medium according to claim 13, wherein the environment information comprises humidity, temperature, and weather.

18. The non-transitory storage medium according to claim 17, wherein the humidity is acquired from a humidity sensor of the electronic device;
the temperature is acquired from a temperature sensor of the electronic device; and
the weather is acquired from internet through a communication device of the electronic device.
